(19) United States
(12) Patent Application Publication
(10) Pub. No.: US 2012/0303007 A1
(43) Pub. Date: Nov. 29, 2012

(54) System and Method for Using Multiple Detectors

(76) Inventors: Frieder Loesel, Mannheim (DE); Roland Toennies, Gernlinden (DE)

(21) Appl. No.: 13/118,027

(22) Filed: May 27, 2011

(51) Int. Cl. A61F 9/008

(52) U.S. Cl. 606/4

(57) ABSTRACT

A system and method are provided for using multiple detectors to create a frame of reference for performing ophthalmic laser surgery. Anatomical detectors generate data sets and a computer program receives these data sets to create the frame of reference. The frame of reference is then used with a selected procedure for conducting ophthalmic laser surgery. An additional detector can measure refractive data of the eye for use as a data set that will refine the frame of reference.

### FIELD OF THE INVENTION

The present invention pertains generally to systems and methods for performing ophthalmic surgery. More particularly, the present invention pertains to performing an ophthalmic procedure using multiple detectors to gather data pertaining to the eye prior to and during the surgery. The present invention is particularly, but not exclusively, useful as a system for planning and performing ophthalmic surgery by combining data gathered by anatomical and optical detector units to develop a three-dimensional frame of reference of the eye.

### BACKGROUND OF THE INVENTION

As is well known, ophthalmic laser surgery can be used to treat a variety of ailments related to the eye. Nearly every part of the eye can benefit from laser-induced changes during ophthalmic surgery to correct various maladies. For instance, ophthalmic laser surgery is commonly used to correct or treat nearsightedness, farsightedness, glaucoma, and cataracts. As would be expected when operating on the human eye, ophthalmic laser surgery is a delicate procedure which must be conducted with the highest degree of care. Mistakes during these types of procedures can have dire consequences to the sight of a patient. More specifically, an improperly directed laser beam can cause significant damage to various areas of the eye and lead to new problems instead of correcting existing ones.

Considering that the risks associated with laser eye surgery are so high, a detailed and precise image of the eye is required during both the planning and execution of an ophthalmic laser procedure. As a consequence, various devices have been developed to create images of the eye for the purpose of guiding and controlling a laser beam during an ophthalmic laser surgery procedure. For instance, simple cameras can be used to create two-dimensional images of an eye. And, more sophisticated devices can be utilized to provide data about internal tissue dimensions. In addition, devices such as wavefront analyzers can be used to determine refractive properties of the eye. Yet, when used individually, many of these devices offer an incomplete frame of reference for the eye. Furthermore, many of the devices do not update an image once an ophthalmic procedure is in progress. This inability to provide updated data can be detrimental because the anatomy of the eye may well undergo significant changes during an ophthalmic procedure. Consequently, the laser eye surgeon may be relying on incomplete or inaccurate data while operating on a patient. When data is inaccurate, the risk of serious damage to the eye of a patient increases significantly.

In light of the above, it is an object of the present invention to provide a system and method for producing a frame of reference for the eye that can be used to plan and execute an ophthalmic laser surgery procedure. Another object of the present invention is to provide a system and method for using multiple detector units to develop a detailed, frame of reference and to then continuously monitor the eye during the procedure using at least one of the detector devices. Yet another object of the present invention is to provide a system and method for using multiple detectors in an ophthalmic laser surgical procedure that is simple to implement, is easy to use, and is comparatively cost effective.

### SUMMARY OF THE INVENTION

In accordance with the present invention, a system and method for using multiple detectors to plan and execute an ophthalmic laser procedure is provided. As contemplated for the present invention, any type of ophthalmic procedure can benefit from the use of multiple detectors. In particular, refractive treatments, corneal treatments, cataract treatments, glaucoma treatments, vitreous treatments, and retinal treatments could all be performed using the systems and methods disclosed here. Prior to commencing the ophthalmic laser procedure, these multiple detectors can be used to develop a precise image of the eye. More specifically, this image of the eye will serve as a three-dimensional frame of reference for the conduct of the laser surgery. Once the procedure begins, at least one of the detectors is used to continuously monitor the eye to provide real-time updates to the frame of reference of the eye being used to guide the procedure.

For the present invention, a laser unit is provided to generate a surgical laser beam that can be used to carry out an ophthalmic laser procedure. This laser unit may also provide a light source for the detectors. In any event, it will also include optics to focus the laser beam at a focal point during the ophthalmic procedure. A controller is connected to a computer and is provided to direct the laser unit during the procedure for this purpose.

Preferably, three separate detector units are provided to obtain both anatomical data and refractive data about the eye. One of the detector units (i.e. a first detector unit) is used to obtain anatomical data about the eye in two-dimensions (x-y directions). As envisioned for the present invention, this can be done by taking a video or a still image of the eye using a camera. Another detector unit (i.e. a second detector unit) is used to obtain additional anatomical data of the eye in a third dimension (z-direction). Imaging methods appropriate for providing this type of third-dimension data include the following: Optical Coherence Tomography (OCT), Scheimpflug imaging, confocal imaging, two-photon imaging, or ultrasound imaging. Together with the two-dimensional image from the first detector unit and z-direction information taken in an orthogonal direction to the two-dimensional image, a three-dimensional frame of reference can be created using data from the first and second detector units. Another detector unit (i.e. a third detector unit) is included in the system of the present invention to provide additional information for the planning of the treatment and for refining the three-dimensional frame of reference. In particular, this third detector unit is preferably a wavefront analyzer that can be used to generate refractive data about the eye. Alternatively, the third detector unit can be used to develop additional structural information about the eye. For instance, instead of a wavefront analyzer, this third detector unit may be an instrument for identifying a corneal topography for the eye, or it may create other types of images that are appropriate for the particular ophthalmic procedure being conducted. For all data sets, a same reference point is identified that can be located anywhere in/on the eye that would be visible in the video or still image produced by the first detector unit. Importantly, all data sets must share at least one common reference point. This is done to ensure all detector units, at least partially, map the same areas (volumes) of the eye, and that these areas (volumes) can be interrelated.

In an operation of the present invention, the plurality of detector units is activated to produce a respective plurality of data sets. Of these, one data set will establish a two-dimensional image of the eye that can be used to identify a reference point and for centration of the laser unit. In detail, centration can occur via one of three ways: (1) automatic pupil detection, (2) detecting a Purkinje reflex, or (3) detecting a reflection from the macula of the eye. Another data set can include measurements that are orthogonal to the two-dimensional image. Together these data sets can be used to produce a three-dimensional frame of reference. As indicated above, yet another data set pertaining to optical characteristics of the eye can be produced to complement and refine the three-dimensional frame of reference. Once all data sets are received at the computer, a computer program compiles all three of the data sets to produce a three-dimensional frame of reference. As noted above, a common reference point is essential to allowing the computer program to line up all data sets for a complete and accurate image of the eye. At this point, a selected procedure can be loaded into the computer for use with the three-dimensional frame of reference. The procedure is then forwarded from the computer to the electronic controller which activates the laser unit. During the procedure, at least one detector unit continues to monitor the eye and update the frame of reference to account for any anatomical or refractive changes induced by the laser procedure.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic diagram of the system for the present invention;

FIG. 2 is a two-dimensional (x-y direction) image of an eye with reference points produced by a detector unit;

FIG. 3 is a diagram illustrating the depth (z-direction) measurements taken using a detector unit; and

FIG. 4 is a graphical representation of the three-dimensional frame of reference.

### DESCRIPTION OF THE PREFERRED EMBODIMENTS

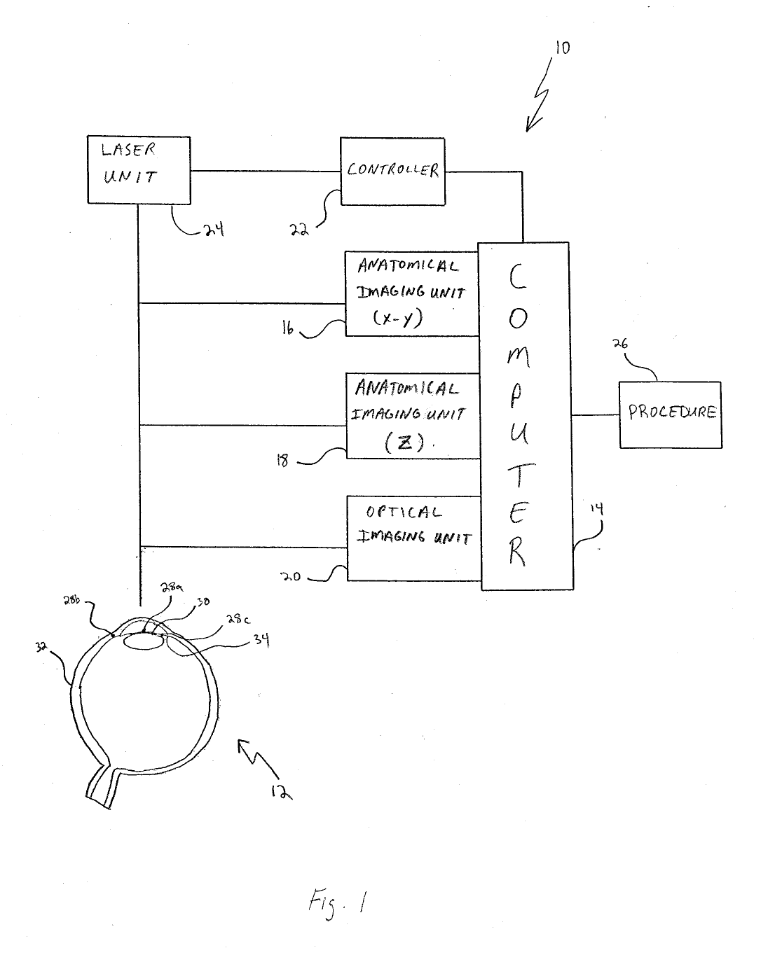

Referring initially to FIG. 1, the system of the present invention is shown and generally designated 10. As depicted, the system 10 is intended for use with a human eye 12 and includes a computer 14 that is in electronic communication with three detector units 16, 18, and 20. Detector unit 16 is an anatomical detector unit that is used to create a two-dimensional (x-y direction) image of the eye 12. For example, the detector unit 16 may be a camera which can produce either a video image or a still image of the eye 12, or both. Also connected to the computer 14 is the detector unit 18 which is used to supplement the two-dimensional image by adding depth data (z-direction). Like detector unit 16, detector unit 18 is also an anatomical detector unit. For the present invention, several different types of detector units 18 can produce an appropriate image for depth data. Examples of these include the following: an OCT imaging unit, a Scheimpflug imaging unit, a confocal imaging unit, a two-photon imaging unit, and an ultrasound imaging unit. An additional detector unit 20 is also included and connected to the computer 14. For purposes of the present invention, detector unit 20 is used to gather refractive data, and, in a preferred embodiment, is a wavefront analyzer. Instead of a wavefront analyzer, detector unit 20 can also be a topographic imaging unit that can be used to form a topographic image of part of the eye 12. As shown, detector unit 20 is integrated with the other two detector units 16 and 18 in the system 10, but it may also be an independent, stand-alone component.

Once data is collected from the three detector units 16, 18, and 20, the computer 14 compiles the data to produce a precise image (three-dimensional frame of reference) for the eye 12. Within this three dimensional frame of reference, a controller 22 is activated to control a laser unit 24 during ophthalmic surgery. For the present invention, the laser unit 24 produces a surgical laser beam to perform laser surgery. In addition, the laser unit 24 may also house an alternate light source for use in conjunction with the detector units to produce the data sets. Importantly, a selected procedure 26 is also loaded into the computer 14 to be transmitted to the controller 22 to perform ophthalmic surgery.

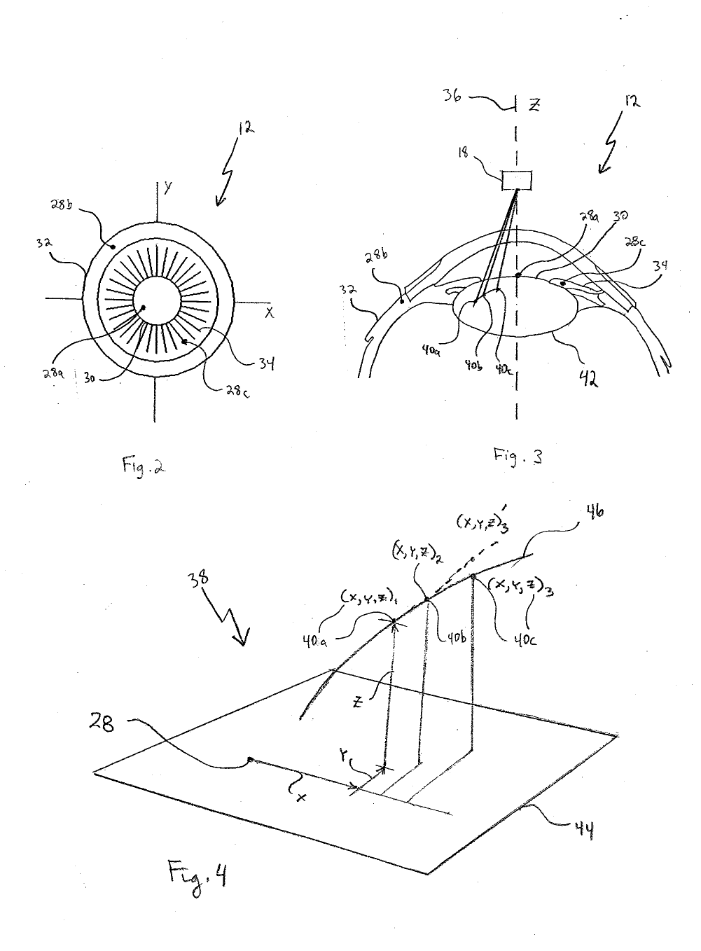

In an operation of the present invention, two-dimensional (x-y) anatomical data is collected using the detector unit 16. Simultaneously, or immediately following the data collection by detector unit 16, detector unit 18 collects data in a third-dimension (z-direction) relative to the two-dimensional (x-y) data. Both data sets include a reference point 28, with the reference point 28 being common to both data sets. These reference points can be established anywhere in the eye that would be visible in a two-dimensional image of the eye 12. As shown in FIG. 1, exemplary reference points 28a-c are located respectively on the pupil 30, the sclera 32, and the iris 34. For cross-reference purposes, the same reference points 28a-c are again shown and included in FIG. 2. In any event, by using a single common reference point, the data sets can be compiled appropriately with a computer program loaded onto the computer 14.

Once each data set is collected, it is electronically transferred to the computer 14. At this point, an initial compilation of data is performed by the computer program to create a three-dimensional frame of reference of the eye 12. This frame of reference may be sufficient for the purposes of the present invention. On the other hand, additional data can be gathered by the detector unit 20 to supplement other data sets. Specifically, supplemental data will preferably concern refractive characteristics of the eye 12. This refractive data set is then sent to the computer 14 to be incorporated into the three-dimensional frame of reference of the eye 12. Importantly, the refractive data set will have at least one reference point in common with the data sets produced by the other two detector units. This common reference point assures the three data sets can be used together to form an accurate frame of reference of the eye 12.

After the three-dimensional frame of reference is produced, a selected procedure 26 is loaded into the computer 14. The selected procedure 26 is used within the context of the three-dimensional frame of reference by the controller 22 to control the laser unit 24 during the ophthalmic procedure. In detail, the procedure 26 includes instructions on moving the focal point of a laser beam to various points within the eye 12 in accordance with the type of procedure being performed.

Referring now to FIG. 3, an illustration is provided to demonstrate the gathering of depth data (z-direction) by detector unit 18. In addition, the three exemplary reference points 28a-c depicted in FIG. 1 and FIG. 2 are also shown. Likewise, the pupil 30, sclera 32, and the iris 34 can also be seen in FIG. 3. In FIG. 3, the visual axis 36 of the eye 12 is shown and serves as the z-axis to illustrate the gathering of data in the z-direction. The concept illustrated in FIG. 3 may be accomplished using any of the following: OCT imaging unit, Scheimpflug imaging unit, confocal imaging unit, two-photon imaging unit, or ultrasound imaging unit.

By cross-referencing FIG. 3 with FIG. 4, an explanation of how the three-dimensional frame of reference 38 is constructed and how the frame of reference 38 is used to conduct ophthalmic laser surgery can also be explained. As shown in FIG. 3, detector unit 18 is used to take depth measurements for three data points 40a-c within the lens 42 of the eye 12 to produce a data set. Stated differently, detector unit 18 is used to provide a z-value for data points 40a-c which already have an x and y value based on the two-dimensional data set produced by detector unit 16. As stated earlier, a reference point 28a-c will also be included in the data set produced by detector unit 18. It should be noted that the lens 42 is used for exemplary purposes, as detector unit 18 can be used to take similar measurements anywhere within the eye 12. In FIG. 4, the same data points 40a-c are shown using x, y, and z-coordinates. For each data point 40a-c, the two-dimensional image 44 includes the x and y-coordinates for each data point 40a-c. Once x and y-coordinates have been established, detector unit 18 establishes the z-values for each data point 40a-c. Importantly, reference point 28 is used by both detector units 16,18 to ensure the frame of reference 38 is constructed properly. It can be seen in FIG. 4 that data point 40a is located at (x,y,z)₁, data point 40b is located at (x,y,z)₂, and data point 40c is located at (x,y,z)₃. In addition, data point 40c′ at (x,y,z)′₃ is a data point produced by detector unit 20 to account for anatomical changes induced during the ophthalmic laser surgery procedure. When grouped together, the data points 40a-c form a path 46. As used for the present invention, this path 46 is followed by the laser beam as the focal point of the laser beam moves from point 40a to point 40c (by way of point 40b) making a cut along the length of the path 46. Such a cut would be commonly used in a procedure such as Laser Induced Optical Breakdown (LIOB). It should be noted that the shape and orientation of the path 46 is only exemplary, and a plurality of data points 40 can be established anywhere within the eye 12 to allow for ophthalmic laser surgery to be performed along any path, surface, or volume of the eye 12.

While the System and Method for Using Multiple Detectors as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A laser system for performing ophthalmic surgery on an eye, wherein the eye has an anatomy defining a reference point, the system comprising:
a laser unit for generating a surgical laser beam, with optics to focus the laser beam to a focal point;
a first detector for generating a first data set pertaining to the anatomy of the eye, wherein the reference point is included in the first data set;
a second detector for generating a second data set pertaining to the anatomy of the eye, wherein the reference point is included in the second data set; and
a computer having a computer program for using the first data set with the second data set to perform a procedure for moving the focal point of the laser beam through the eye for the conduct of the ophthalmic surgery.

2. A system as recited in claim 1 wherein the first data set creates a two-dimensional (x,y) image of the eye.

3. A system as recited in claim 2 wherein the first data set is used for orienting the laser unit on the eye prior to using the procedure for moving the focal point of the laser beam.

4. A system as recited in claim 2 wherein the first data set is selected from a group comprising video data and still image data.

5. A system as recited in claim 2 wherein the second data set includes linear depth measurements (z-direction) taken relative to the two-dimensional image of the first data set, wherein the depth measurements are respectively taken along a plurality of parallel lines, and wherein each line is substantially perpendicular to the two-dimensional image of the first data set, and further wherein each line is at a known location relative to the reference point in the two-dimensional image of the first data set.

6. A system as recited in claim 5 wherein the second data set is generated by a process selected from a group comprising Optical Coherence Tomography (OCT), Scheimpflug, confocal imaging, two-photon imaging, and ultrasound imaging.

7. A system as recited in claim 1 wherein the reference point on the anatomy of the eye is selected from a group comprising points on the pupil, the iris, and the sclera.

8. A system as recited in claim 1 further comprising a third detector for generating a third data set pertaining to optical characteristics of the eye, wherein the reference point is included in the third data set.

9. A system as recited in claim 8 wherein the optical characteristics of the eye are selected from a group comprising refractive properties, interference patterns, and diagnosed optical defects.

10. A system as recited in claim 8 wherein the computer program uses the third data set with the first and second data sets to establish the procedure for moving the focal point.

11. A method for performing ophthalmic laser surgery on an eye, wherein the eye has an anatomy defining a reference point, the method comprising the steps of:
electronically connecting a computer with a laser unit, wherein the laser unit generates a surgical laser beam, and wherein the laser unit has optics to focus the laser beam to a focal point;
generating a first data set pertaining to the anatomy of the eye using a first detector, wherein the reference point is included in the first data set;
generating a second data set pertaining to the anatomy of the eye using a second detector, wherein the reference point is included in the second data set; and
creating a predetermined computer program for using the first data set with the second data set to establish a procedure for moving the focal point of the laser beam through the eye for the conduct of the ophthalmic surgery.

12. A method as recited in claim 11 wherein the first data set creates a two-dimensional (x,y) image of the eye.

13. A method as recited in claim 11 wherein the first data set is used to orient the laser unit on the eye prior to use.

14. A method as recited in claim 11 wherein the second detector obtains data in a third dimension (z) relative to the two-dimensional (x,y) image of the eye created by the first data set.

15. A method as recited in claim 11 further comprising the step of:
generating a third data set pertaining to the optical characteristics of the eye, wherein the reference point is included in the third data set.

16. A method as recited in claim 15 wherein the predetermined computer program uses data from the first data set, the second data set, and the third data set to establish the procedure for moving the focal point of the laser beam through the eye.

17. A system for performing ophthalmic laser surgery on an eye, wherein the eye has an anatomy defining a reference point, the system comprising:
a laser unit for generating and focusing a laser beam at a focal point in the eye;
a computer connected to the laser unit for controlling the laser beam;
a plurality of detectors for generating a respective plurality of data sets, wherein each data set incorporates the reference point, and the plurality of data sets establishes a three-dimensional frame of reference in the eye;
a selected procedure for performing the ophthalmic laser surgery; and
a computer program for use by the computer, wherein the computer program receives the selected procedure and the plurality of data sets as input for collective use in performing the ophthalmic laser surgery.

18. A system as recited in claim 17 wherein a first data set in the plurality of data sets includes data in two dimensions.

19. A system as recited in claim 18 wherein a second data set in the plurality of data sets includes data in a third dimension.

20. A system as recited in claim 17 wherein a third data set in the plurality of data sets pertains to optical characteristics of the eye.
